(19) United States
(12) Patent Application Publication
(10) Pub. No.: US 2001/0048946 A1
(43) Pub. Date: Dec. 6, 2001

(54) SOLID PHARMACEUTICAL DOSAGE FORMS IN FORM OF A PARTICULATE DISPERSION

(76) Inventor: ISAAC GHEBRE-SELLASSIE, MORRIS PLAINS, NJ (US)

Correspondence Address: CHARLES W ASHBROOK, WARNER LAMBERT COMPANY, 2800 PLYMOUTH ROAD, ANN ARBOR, MI, 48105

(21) Appl. No.: 09/284,858

(22) PCT Filed: Jul. 29, 1998
(86) PCT No.: PCT/US98/15693

§ 371 Date: None

(30) Foreign Application Priority Data
Aug. 21, 1997 (US) 60/056195

(51) Int. Cl. A61K 9/14; A61K 9/16; A61K 9/50

(52) U.S. Cl. 424/486; 424/494; 424/497

(57) ABSTRACT

Solid particulate dispersions of pharmaceutical agents in a matrix of a water-soluble polymer exhibiting good aqueous dissolution and enhanced bioavailability.

### FIELD OF THE INVENTION

This invention relates to orally bioavailable solid dosage forms of poorly water-soluble pharmaceutical agents.

### BACKGROUND OF THE INVENTION

Many pharmaceutical agents are such highly complex chemical structures that they are insoluble or only sparingly soluble in water. This results in no or very low dissolution from conventional dosage forms designed for oral administration. Low dissolution rates results in no or very little bioavailability of the active chemical substance, thus making oral delivery ineffective therapeutically, and necessitating parenteral administration in order to achieve a beneficial therapeutic result. Drug products that are limited to parenteral delivery leads to increased costs of medical care, due to higher costs of manufacturing, more costly accessories required for delivery, and in many cases hospitalization of the patient to ensure proper dosing (e.g., sterile intravenous delivery).

Poorly water-soluble drugs that undergo dissolution rate-limited gastrointestinal absorption generally show increased bioavailability when the rate of dissolution is improved. To enhance the dissolution property and potentially the bioavailability of poorly water-soluble drugs, many strategies and methods have been proposed and used, which include particle size reduction, salt selection, formation of molecular complexes and solid dispersions, and the use of metastable polymorphic forms, co-solvents, and surface-active agents. Of these methods, the use of surface-active agents is mainly to improve the wettability of poorly water-soluble drugs, which eventually results in the enhancement of the rate of dissolution.

We have now discovered a method for producing solid particulate dosage forms of poorly water-soluble pharmaceutical agents, making them ideally suited for oral administration, and providing enhanced dissolution rate in water and hence improved oral bioavailability. The method of this invention utilizes water-soluble polymers such as polyvinylpyrrolidone, hydroxypropyl cellulose, or hydroxypropyl methylcellulose as carriers. The use of these water-soluble carriers improves the wettability of the poorly water-soluble crystalline pharmaceutical agents, thus improving the rate of their dissolution following administration, and finally resulting in improved bioavailability and therapeutic result. The invention provides for mixing or extruding the active ingredients in solid particulate form with the polymeric carrier at a temperature at which the polymer softens, or even melts, but the drug remains solid or crystalline. The drug particles thus become coated and produce a product that is matrix coated, i.e., a particulate dispersion.

### SUMMARY OF THE INVENTION

This invention provides solid dosage forms of sparingly water-soluble pharmaceutical agents. More particularly, the invention is a pharmaceutical composition in the form of a solid particulate dispersion of a particulate pharmaceutical ingredient dispersed throughout a matrix of a water-soluble polymer such as polyvinylpyrrolidone, hydroxypropyl cellulose, or hydroxypropyl methylcellulose.

In a preferred embodiment, the particulate pharmaceutical ingredient is dispersed in a water-soluble polymer in a weight ratio of about 10% to about 90% active ingredient to about 90% to about 10% polymer. A preferred formulation comprises about 20% to about 80% of active ingredient and about 80% to about 20% polymer. The most preferred composition comprises about 50% to about 80% solid active ingredient, and about 20% to 50% polymer or other excipients.

In another preferred embodiment, the pharmaceutical ingredient is dispersed in hydroxypropyl cellulose or hydroxypropyl methylcellulose. Especially preferred compositions comprise 40% to 80% by weight of active ingredient. The precise ratio of polymer to drug in the matrix is dictated by the particle size, and thus the surface area of the crystalline drug substance. Other conventional excipients such as glycerin, propyleneglycol, Tween, stearic acid salts, polyvinyl pyrrolidones and the like can be added.

In an especially preferred embodiment, the sparingly soluble pharmaceutical agent utilized is selected from the class known as the glitazones. The glitazones are thiazolidinedione antidiabetic agents such as troglitazone, ciglitazone, pioglitazone, englitazone, and BRL 49653.

The most preferred composition of the invention is a solid dispersion of troglitazone in hydroxypropyl cellulose.

### DETAILED DESCRIPTION OF THE INVENTION

The compositions provided by this invention are particulate dispersions of sparingly soluble pharmaceutical agents in a water-soluble polymer such as hydroxypropyl cellulose or hydroxypropyl methylcellulose.

Hydroxypropyl cellulose is also known as cellulose 2-hydroxypropyl ether, oxypropylated cellulose, and HPC. It is a non-ionic water-soluble ether of cellulose which exists as an off-white powder. While hydroxypropyl cellulose is soluble in many polar organic solvents, it readily precipitates from water at about 40° C. It is a thermoplastic material that has been utilized in the pharmaceutical field as an emulsifier, stabilizer, whipping aid, protective colloid, as well as a film former or thickener in foods.

Hydroxypropyl methylcellulose is cellulose 2-hydroxypropyl methyl ether or HPMC. It is a non-ionic water-soluble ether of methylcellulose, which is insoluble in hot water but dissolves slowly in cold water. It is more soluble than methylcellulose, and has been used extensively as an emulsifier, stabilizer, suspending agent, tablet excipient, and most notably as an ophthalmic lubricant. It is sold commercially as Ultra Tears, Tearisol, and Goniosol.

The compositions of this invention employ sparingly soluble pharmaceutical agents. The term “sparingly soluble pharmaceutical agent” means any solid or crystalline drug substance 1 gram of which will dissolve in from 30 to 100 grams of water at 25° C. Numerous drug substances are “sparingly soluble pharmaceutical agents” as used herein, and can be employed to make the particulate dispersions of this invention. As noted above, a preferred group of such agents are the glitazones, especially troglitazone, also known as “CI-991”. The glitazones are described more fully in U.S. Pat. No. 5,478,852, which is incorporated herein by reference. Other agents that can be employed include antibiotics, such as cephalosporins and penicillins, the fluoroquinolinones such as clinafloxacin, the naphthyridinones such as CI-990, and the erythromycyl amine type compounds. Antihypertensive agents such as chlorothiazide and the ACE-inhibitors (quinapril, vasotec) can be formulated according to this invention. Anticancer agents such as methotrexate, suramin, and the vinca alkaloids can be employed.

Other pharmaceuticals which can be formulated as particulate dispersions include, but are not limited to acetohexamide, ajamaline, amylobarbitone, bendrofluazide, benzbromarone, benzonatate, benzylbenzoate, betamethazone, chloramphenicol, chlorpropamide, chlorthalidone, clofibrate, corticosteroids, diazepam, dicumerol, digitoxin, dihydroxypropyltheophylline, ergot alkaloids, ethotoin, frusemide, glutethimide, griseofulvin, hydrochlorothiazide, hydrocortisone, hydroflumethiazide, hydroquinone, hydroxyalkylxanthines, indomethacin, isoxsuprine hydrochloride, ketoprofen, khellin, meprobamate, nabilone, nicotainamide, nifedipine, nitrofurantoin, novalgin, nystatin, papaverine, paracetamol, phenylbutazone, phenobarbitone, prednisolone, prednisone, primadone, reserpine, romglizone, salicylic acid, spiranolactone, sulphabenzamide, sulphadiamadine, sulphamethoxydiazine, sulphamerazine, succinylsulphathiazole, sulphamethizole, sulphamethoxazole, sulphathiazole, sulphisoxazole, testosterone, tolazoline, tolbutamide, trifluoperazine, trimethaprim, and other water-insoluble drugs.

Any number of water-soluble polymers can be employed as a carrier for the particulate dispersion. All that is required is that the polymer be capable of softening or melting at a temperature that does not melt the solid drug substance, so that a matrix coating on the particulate drug substance can be formed. The polymer also must be sufficiently water soluble to allow dissolution of the particulate dispersion at a rate that provides the desired oral bioavailability and resulting therapeutic benefit. Typical polymers to be employed include polyvinylpyrrolidone (PVP), polyethylene-oxides, pregelatinized starch, methylcellulose, hydroxyethylcellulose, polyvinyl alcohol, sodium alginate, sodium carboxymethylcellulose, lecithin, tweens, maltodextrin, poloxamer, sodium laurylsulfate, polyethylene glycol (PEG), vinyl acetate copolymer, Eudragit® acrylic polymers, E-100, and mixtures thereof. The carrier of choice obviously is dependent upon the drug to be dispersed but generally, the chosen carrier must be pharmacologically inert and chemically compatible with the drug in the solid state. They should not form highly bonded complexes with a strong association constant and most importantly should be freely water soluble with intrinsic rapid dissolution properties.

Another polymer of choice in most dispersions is PVP, which is a free flowing amorphous powder that is soluble in both water and organic solvents. It is hygroscopic in nature and compatible with a wide range of hydrophilic and hydrophobic resins. Another preferred carrier is a high molecular weight polyethylene glycol such as PEG 6000, which is a condensation polymer of ethylene glycol. Polyethylene glycols are generally a clear, colorless, odorless viscous liquid to waxy solid that is soluble or miscible with water.

The surprising and unexpected results of the present invention is the creation of a solid particulate pharmaceutical dispersion comprised of the aforementioned water-insoluble drugs and carriers without the need for using aqueous or organic solvents. In a further embodiment, the addition of a plasticizer/solubilizer during the mixing of the particulate drug and water-soluble polymer results in a chemical environment that readily lends itself to particulate dispersion formation.

Suitable plasticizers/solubilizers useful in the practice of the present invention include low molecular weight polyethylene glycols such as PEG 200, PEG 300, PEG 400, and PEG 600. Other suitable plasticizers include propylene glycol, glycerin, triacetin, and triethyl citrate. Optionally, a surfactant such as Tween 80 may be added to facilitate wettability within the formulation.

The water-insoluble drug of interest can first be milled to the desired particulate size, generally from about 1 micron to about 20 microns. It then is blended with the polymeric carrier using any appropriate mixer or blender in a drug/carrier ratio of from about 1:9 to about 5:1, respectively, based upon a percentage weight basis. Preferably, the drug/carrier ratio will be approximately 3:1 to about 1:3, respectively. The blend is then transferred to a mixer, for example a low or high shear mixer or a fluid bed granulator, and additional excipients can be added, for example a plasticizer such as PEG 400, which can be dissolved in water with a surfactant such as Tween 80, if desired. Other suitable surfactants include Tweens 20 and 60, Span 20, Span 40, Pluronics, polyoxyethylene sorbitol esters, monoglycerides, polyoxyethylene acids, polyoxyethylene alcohols and mixtures thereof. Once all ingredients are sufficiently dissolved or suspended, the solution is sprayed onto the powder blend in the fluid bed granulator under specific conditions. The mixture can also be granulated in a low or high shear mixer, dried, and molded to produce the granulated product. The resultant granulation is transferred to a container and fed into a high intensity mixer such as a twin-screw extruder with at least one, and preferably more than one heating zones. The mixture is then extruded at appropriate temperatures depending on the heat stability of the drug, until a particulate dispersion is collected as an extrudate, which is then transferred to a drum for milling. The milled particulate pharmaceutical dispersion can then be ground into a powdery mass, and further blended with other excipients prior to encapsulation or being pressed into tablets. The final dosage form by may be optionally coated with a film such as hydroxypropyl methylcellulose, if desired.

In a preferred embodiment, particulate dispersions of the invention are prepared by melt extrusion of a pharmaceutical agent and about 10 to 90 weight percent of a polymer such as HPC. The melt extrusion is carried out by mixing the ingredients to uniformity at a temperature of about 50° C. to about 200° C., the temperature being sufficiently high to melt or soften the polymer, but not so high to melt the drug particles. The melt or softened mixture is passed through a commercial twin-screw extruder. The resulting extrudate can be employed directly, or can be further processed, for example by milling or grinding to the desired consistency, and further admixed with conventional carriers such as starch, sucrose, talc and the like, and pressed into tablets or encapsulated. The final dosage forms generally will contain about 1 mg to about 1000 mg of active ingredient, and more typically about 300 mg to about 800 mg.

### BRIEF DESCRIPTION OF FIGURES

FIG. 1 is the X-ray powder diffractogram of bulk troglitazone (CI-991).

FIG. 2 is the X-ray powder diffractogram of the particulate dispersion of CI-991 in PEG-8000 and PVP in a weight ratio of 80:10:10.

FIG. 3 is the X-ray powder diffractogram of the particulate dispersion of CI-991 in PEG-8000 and HPC in a weight ratio of 80:10:10.

FIG. 4 is the X-ray powder diffractogram of the particulate dispersion of CI-991 in PEG-8000 and PVP in a weight ratio of 75:10:15.

FIG. 5 is the X-ray powder diffractogram of the particulate dispersion of CI-991, PEG-8000, and HPC in the weight ratio of 75:10:15.

FIG. 6 is the X-ray powder diffractogram of the particulate dispersion of CI-991, PEG-8000, and HPC in the weight ratio of 75:5:20.

FIG. 7 is the X-ray powder diffractogram of the particulate dispersion of CI-991, and HPC in the weight ratio of 75:25.

FIG. 8 is a comparison of dissolution profiles at pH 8 for various particulate dispersion formulations of CI-991.

FIG. 9 is a comparison of dissolution profiles at pH 9 for various particulate dispersion formulations of CI-991.

FIG. 10 is a comparison of dissolution profiles at pH 8 for two formulations of CI-991 in PVP.

FIG. 11 is a comparison of dissolution profiles at pH 9 for two formulations of CI-991 in PVP.

FIG. 12 is a comparison of dissolution profiles at pH 8 of various particulate dispersion formulations of CI-991.

The following detailed examples further illustrate the present invention. The examples are illustrative only and should not be construed to limit the invention in any respect.

### EXAMPLE 1

### Particulate Dispersion of Chlorothiazide

A mixture of 54 g of chlorothiazide and 6 g of hydroxypropyl cellulose were blended to uniformity at 24° C. using a mortar and pestal. The mixture was transferred to a rotating mixing bowl and heated to 150° C., and tumbled at 50 rpm. The torque was maintained at 2000 meter-grams. The mixture congealed, and upon cooling to 24° C., was solid and uniform. The product was pulverized and milled, and pressed into tablets. Each tablet was a solid particulate formulation of chlorothiazide.

### EXAMPLE 2

A mixture of 54 g of chlorothiazide and 6 g of hydroxypropyl methylcellulose were blended to uniformity at 24° C. in a mortar and pestal. The mixture was added to a rotating mixing bowl and blended for 1 hour at 170° C. at 50 rpm. The mixture was cooled, milled, and pressed into tablets which were solid particulate dispersions of chlorothiazide.

### EXAMPLE 3

Troglitazone (CI-991), a new drug developed for the treatment of noninsulin-dependent diabetes, is a practically water-insoluble drug in gastrointestinal pH range of 1.0 to 7.5. To date, CI-991 has been prepared as a solid dispersion, in which the crystalline drug substance is converted to the amorphous form by hot melt extrusion methods, to enhance its rate of dissolution and oral bioavailability. In this study, CI-991 was used as a model drug to test whether the dissolution rate of poorly water-soluble drugs could be enhanced by the approach of forming a particulate dispersion in a matrix of a water-soluble polymer.

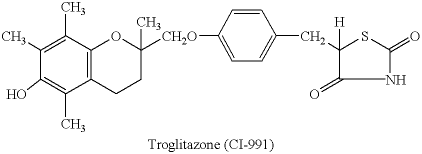

### Materials

CI-991 bulk drug (Lot XX020195) and the selected water-soluble excipients, including HPC, PVP K28-32, and PEG-8000, were all obtained from Centralized Raw Materials (Morris Plains, N.J.). Chemicals used for preparing dissolution media, including disodium hydrogen phosphate (Na₂HPO₄), dipotassium hydrogen phosphate (K₂HPO₄), and 85% phosphoric acid (H₃PO₄), were obtained from J. T. Baker Co. (Phillisburg, N.J.), whereas sodium lauryl sulfate (SLS) was obtained from Centralized Raw Materials.

### Preparation of CI-991 Particulate Dispersions (PD)

CI-991 particulate dispersions were prepared by the mixing bowl method. The appropriate weights of CI-991 and excipients were placed in a screw-capped bottle and blended by a turbula mixer (Glen Mills Co., Maywood, N.J.) for 15 minutes to give powder blends (or physical mixtures). About 65 grams of the powder blends were then mixed in a Brabender twin-screw mixing bowl (C. W. Brabender Instruments, South Hackensack, N.J.) at 110° C. or 130° C. for 5 minutes. The resulting products (CI-991 PD) were collected, milled, and sieved. Samples having particle size between 80- and 100-mesh were used for dissolution study and other tests.

### HPLC Assay of CI-991 Particulate Dispersions

The HPLC method used for the assay of CI-991 was adopted from RTD-0991-TAC-5 (pp. 5-12). HPLC analysis was conducted on a Hewlett-Packard 1090 HPLC system equipped with a Hewlett-Packard 1050 absorbance detector and an Alltech Hypersil C18 column (4.6×100 mm, 3 μm). The mobile phase consisted of a 50:50 (% v/v) mixture of pH 3 (0.05 M) triethylamine buffer and acetonitrile. The flow rate was 1.5 mL/min, the UV detection wavelength was 225 nm, the injection volume was 20 82 L, and the run time was 15 minutes. The retention time for the CI-991 peak was found to be around 5.6 minutes. Data acquisition and integration was performed with a Hewlett-Packard ChemStation software (Rev. A.02.00).

### Characterization of Crystallinity

Crystallinity of the CI-991 particulate dispersions was characterized using X-ray powder diffractometry. X-ray powder diffraction patterns were recorded by using a Rigaku Geiger-Flex X-ray Diffractometer with Ni-filtered Cu-Kα radiation (λ=1.5418 Å) over the interval 4-40°/2θ. In some cases, polarizing optical microscopy was used to confirm the results obtained from X-ray powder diffraction. The microscopic investigation was conducted in a Leitz Labolux 12 polarizing optical microscope equipped with a Polaroid camera.

### Dissolution Studies

### Preparation of Dissolution Media

### pH 8 (0.1 M) Phosphate Buffer Containing 0.5% (g/mL) SLS

(0.1 M) Phosphate solution was prepared by dissolving a calculated amount of Na₂HPO₄ in USP water. The pH-value of the (0.1 M) phosphate solution was then adjusted to 8.0±0.02 by 85% phosphoric acid to give a pH 8 (0.1 M) phosphate buffer. An appropriate amount of SLS was added and dissolved in the pH 8 (0.1 M) phosphate buffer to give the pH 8 (0.1 M) phosphate buffer containing 0.5% (g/mL) SLS.

### pH 9 (0.05 M) Phosphate Buffer

(0.05 M) Phosphate solution was prepared by mixing 1:1 ratio of the aqueous solutions of (0.025 M) Na₂HPO₄ and (0.025 M) K₂HPO₄. The pH value of the (0.05 M) phosphate solution was then adjusted to 9.0±0.02 by 85% phosphoric acid to give the pH 9 (0.05 M) phosphate buffer.

### Dissolution Testing

The dissolution studies were conducted in 900 mL of dissolution medium maintained at 37° C., using USP apparatus II (Distek 2100A dissolution system, North Brunswick, N.J.) at 75 rpm of paddle speed. After dispersing a sample containing 100 mg of CI-991 into the dissolution medium, about 10 mL of solutions were periodically sampled and filtered by Gelman Nylon Acrodisc 0.45 μm filters to give clear filtrates (discard the first 2 mL filtrate). The extent of the drug dissolved in the dissolution medium was determined by UV spectrometry at λ=284 nm. Interference by the excipients was not observed during analysis. Experiments were run in duplicate, and the results were averaged.

### RESULTS AND DISCUSSION

### Preparation and HPLC Assay of CI-991 Particulate Dispersions

Depending on sample sizes, particulate dispersion could be prepared by the mixing bowl or extrusion method. To minimize the quantity of CI-991 bulk drug utilized, CI-991 particulate dispersions were prepared using the mixing bowl method in this exploratory study. Since the melting range of CI-991 has been reported as 165° C. to 175° C., the temperature applied to the mixing process should be lower than the melting temperature of CI-991 to prevent the drug from melting but should be high enough to soft or melt the water-soluble excipients used. By using this mixing bowl method, six CI-991 particulate dispersions, namely CI-991/PEG-8000/PVP (80:10:10), CI-991/PEG-8000/HPC (80:10:10), CI-991/PEG-8000/PVP (75:0:15), CI-991/PEG-8000/HPC (75:10:15), CI-991/PEG-8000/HPC (75:5:20), and CI-991/HPC (75:25) PD, were prepared at 110° C. or 130° C. [Table 1].

To investigate the chemical stability of CI-991 during the mixing process, the six CI-991 particulate dispersions were assayed using HPLC method. As presented in Table 1, the contents of drug measured from the six CI-991 particulate dispersions all agree well with those of the theoretical values, suggesting that CI-991 did not decompose significantly as the drug was mixed with PEG, HPC, and/or PVP at 110° C. or 130° C.

TABLE 1
Preparation and HPLC Assay of Various CI-991/Polymer
Particulate Dispersions (PD)

| | | Percision | | |
|---|---|---|---|---|
| | Formulation of | Temper- | Percent of CI-991 | |
| Sample | CI-991 Particulate | ature | Theoretical | Assayed |
| ID | Dispersions | ° C. | (%) | (%) |
| TD-0921096 | CI-991/PEG- | 110 | 80 | 78.42 ± 0.33 |
| | 8000/PVP | | | |
| | (80:10:10) | | | |
| TD-0931096 | CI-991/PEG- | 110 | 80 | 78.41 ± 0.11 |
| | 8000/HPC | | | |
| | (80:10:10) | | | |
| TD-0941096 | CI-991/PEG- | 130 | 75 | 73.98 ± 0.12 |
| | 8000/PVP | | | |
| | (75:10:15) | | | |
| TD-0951096 | CI-991/PEG- | 130 | 75 | 73.79 ± 0.02 |
| | 8000/HPC | | | |
| | (75:10:15) | | | |
| TD-0961096 | CI-991/PEG- | 130 | 75 | 73.61 ± 0.05 |
| | 8000/HPC | | | |
| | (75:2:20) | | | |
| TD-0971096 | CI-991/HPC | 130 | 75 | 74.13 ± 0.24 |
| | (75:25) | | | |

### X-ray Powder Diffraction Study

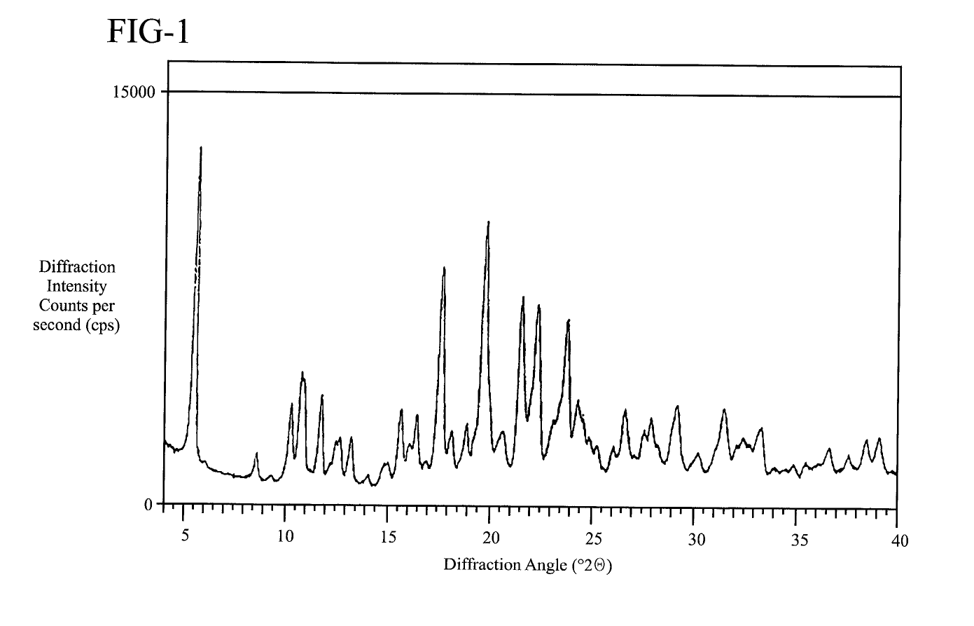

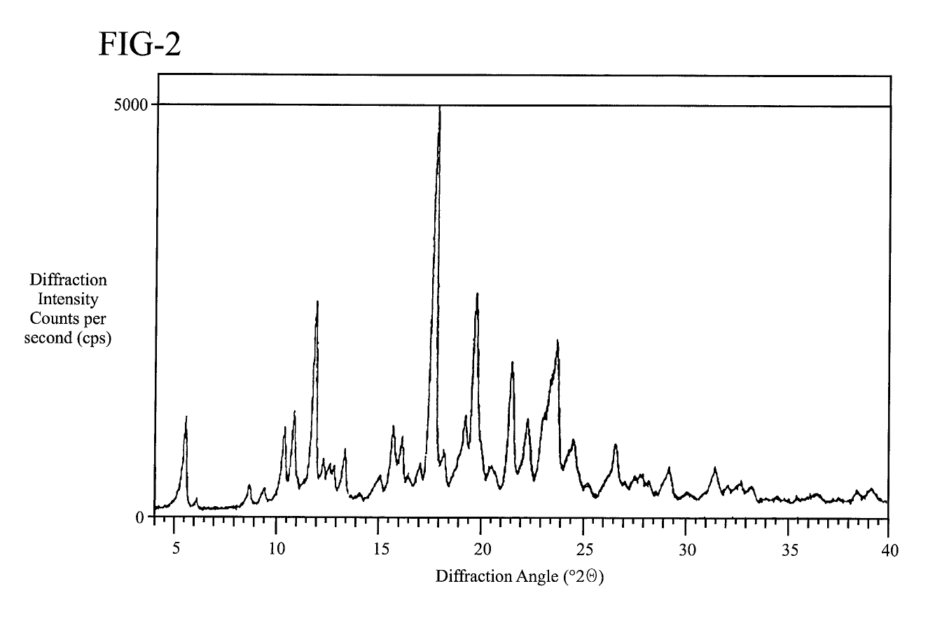

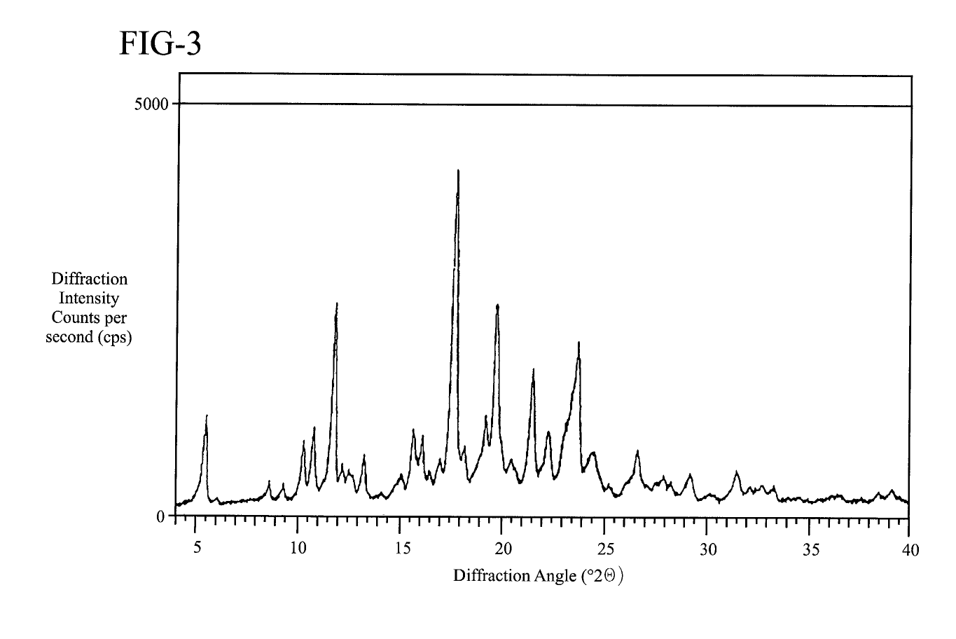

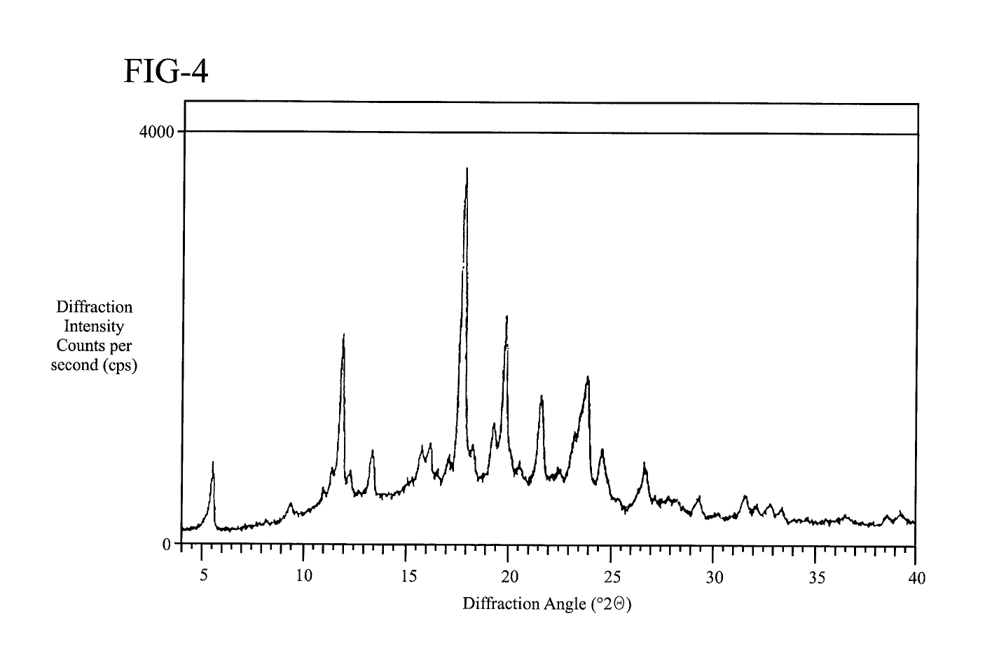

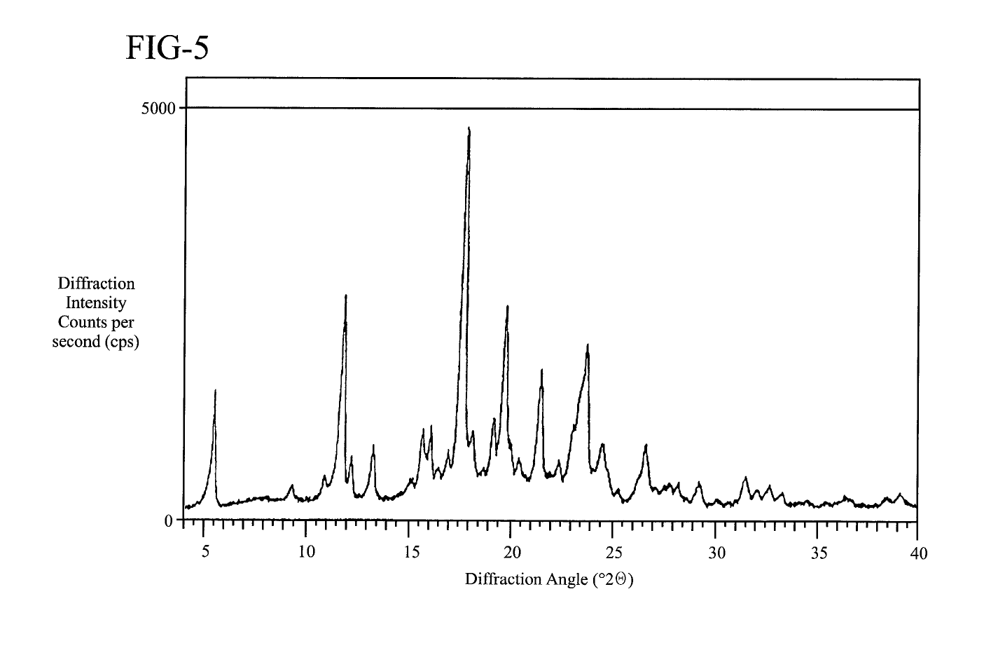

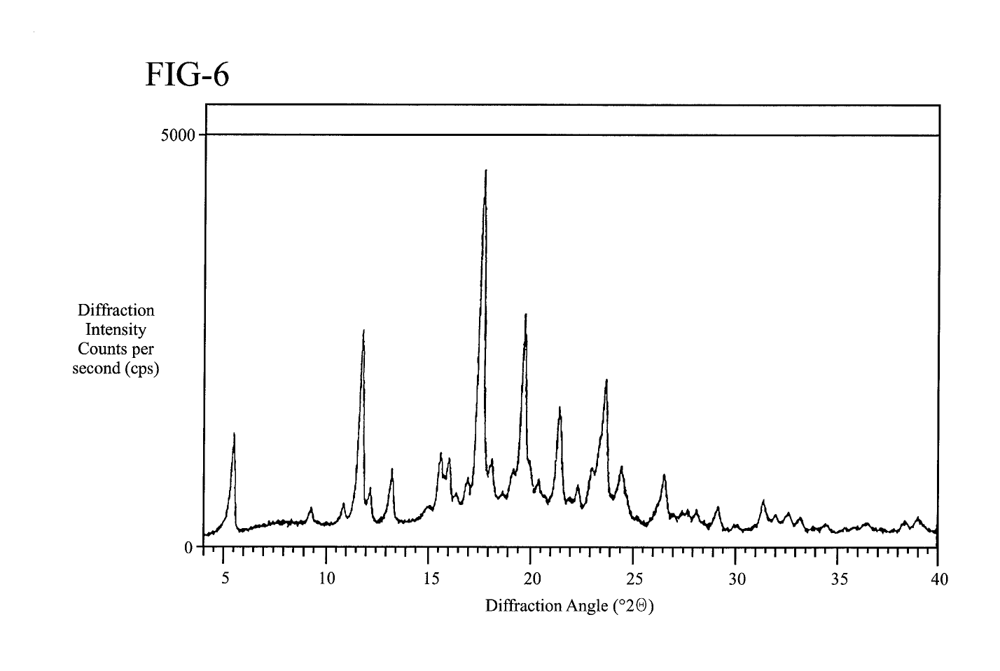

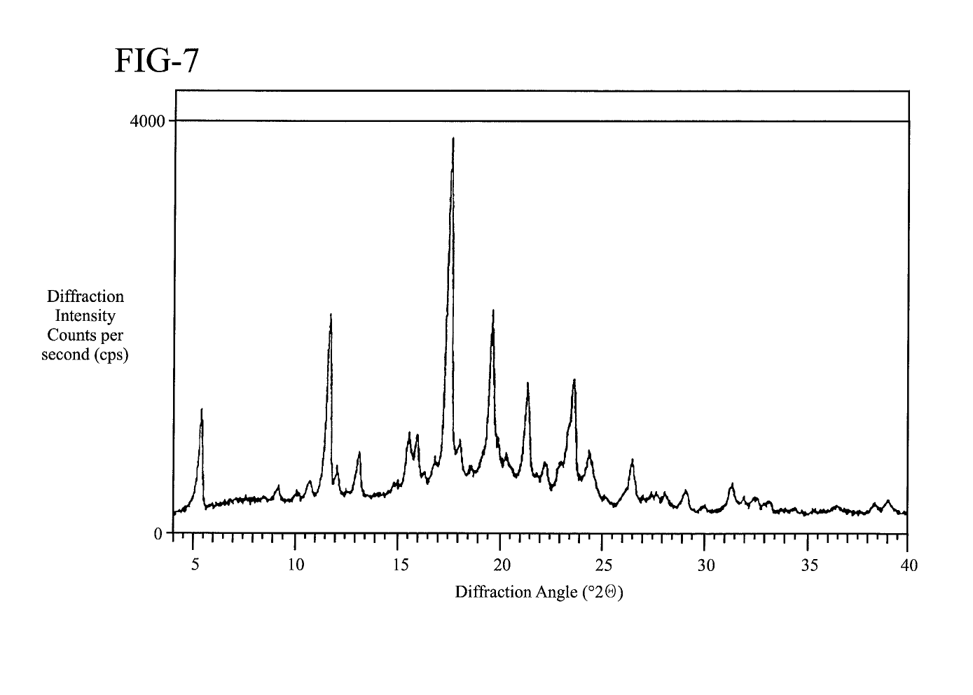

Since the mixing temperature (110 or 130° C.) is well below the melting range of CI-991 (165-175° C.), the drug is not expected to melt or convert to amorphous form during the formation of CI-991 particulate dispersion. The X-ray powder diffraction patterns of the CI-991 bulk drug and the six CI-991 particulates are shown in FIG. 1 and in FIGS. 2-7, respectively. The crystalline properties of the bulk drug are characterized by several major diffraction peaks near 5.5, 11.8, 17.6, 19.6 and 23.7° (2θ), in the diffractogram [FIG. 1]. For CI-991/PEG/PVP and CI-991/PEG/HPC (80:10:10) PD that were prepared at 110° C., their X-ray diffraction patterns [FIGS. 2-3] are almost identical to that of CI-991 bulk drug. Except a few weak diffraction peaks in the region of 8.5-0.5 2θ), most identifiable diffraction peaks of CI-991 are observed in the diffractograms of CI-991/PEG/PVP (75:10:15), CI-991/PEG/HPC (75:10:15), CI-991/PEG/HPC (75:5:20) and CI-991/HPC (75:25) PD [FIGS. 4-7], which were prepared at 130° C. FIGS. 1-7 also revealed that the CI-991 particulate dispersions, especially for those prepared at 130° C., exhibited broader diffraction peaks than the CI-991 bulk drug. These data may indicate that the crystalline bulk drug has been partially converted to the amorphous form and/or interacts with the polymers used during the mixing process at elevated temperatures for the preparation of CI-991 particulate dispersions.

### Dissolution Studies

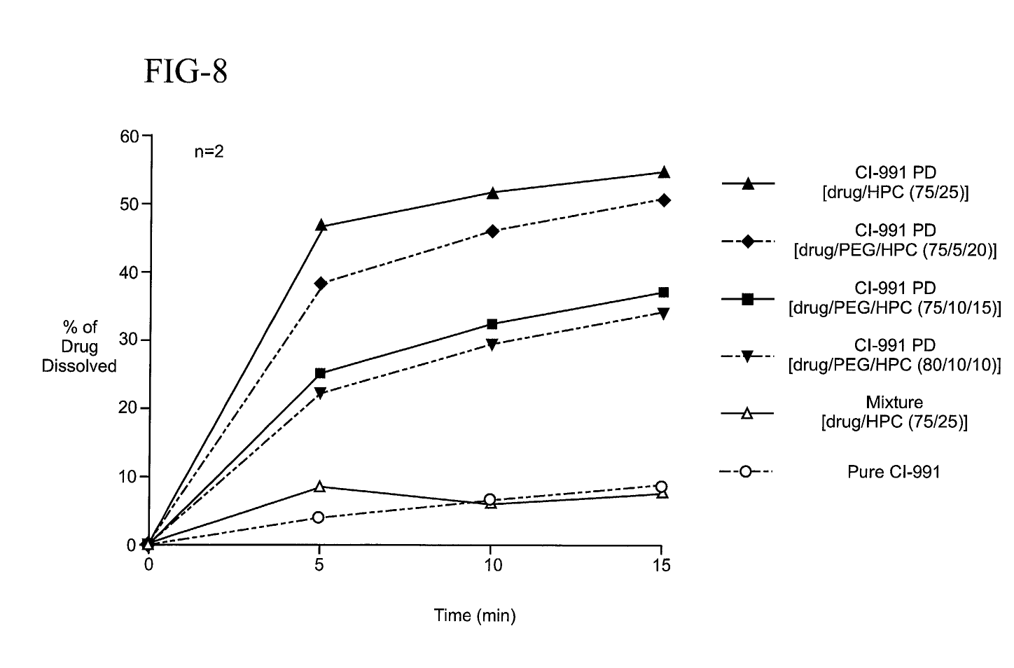

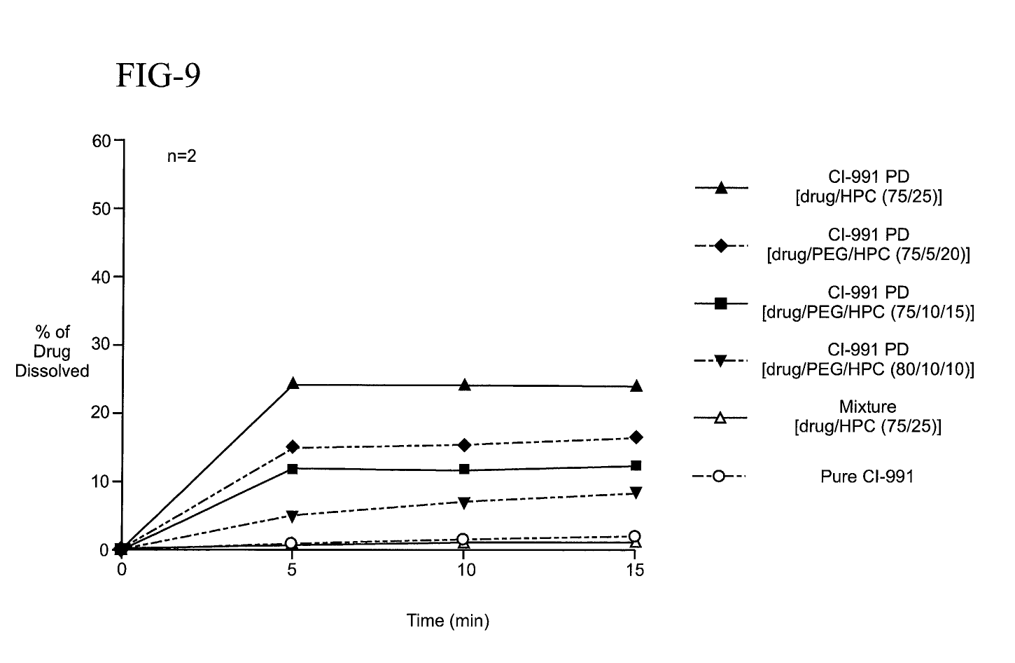

The dissolution behaviors of the CI-991/polymer particulate dispersions were studied in two different dissolution media, namely pH 8 (0.1 M) phosphate buffer containing 0.5% SLS and pH 9 (0.05 M) phosphate buffer. The dissolution profiles of various CI-991 /PEG-8000/HPC particulate dispersions in pH 8 (0.1 M) phosphate buffer containing 0.5% SLS and in pH 9 (0.05 M) phosphate buffer are shown in FIGS. 8 and 9, respectively. The dissolution profiles of the CI-991 bulk drug (or pure CI-991) and CI-991/HPC (75:25) physical mixture are also shown in FIGS. 8 and 9 for comparison.

It clearly indicates that all the four CI-991/HPC particulate dispersions exhibit a greater rate and extent of dissolution of CI-991 than the pure drug and physical mixture in these two dissolution media. The enhancement of dissolution rates of CI-991 would be mainly due to the increase of wettability of CI-991, since the drug has been coated with HPC and/or PEG-8000 (water-soluble polymers) during the formation of CI-991 particulate dispersion. In addition to the coating of water-soluble polymers, the rate of dissolution of CI-991 could be enhanced by the reduction of particle size since the drug might have been finely dispersed in the matrix of the polymers during the mixing process.

Of the four particulate dispersions studied, CI-991/HPC (75:25) PD exhibited the highest rate of dissolution. This is understandable because this particulate dispersion has the highest concentration of HPC, in which the resulting particulates would have the best wettability of the four CI-991 /HPC particulate dispersions. The CI-991/HPC (75:25) PD yielded a 12-fold greater initial dissolution rate (computed over the first 5 minutes of dissolution) in pH (0.1 M) phosphate buffer containing 0.5% SLS than the pure CI-991 (Table 2 and FIG. 8). In pH 9 (0.05 M) phosphate buffer, CI-991/HPC (75:25) PD also yielded a much greater initial dissolution rate than the pure CI-991 (Table 2 and FIG. 9). After 15 minutes, this particulate dispersion produced a 7-fold greater dissolution rate in pH 8 (0.1 M) phosphate buffer containing 0.5% SLS and a 20-fold greater dissolution rate in pH 9 (0.05 M) phosphate buffer than the pure drug.

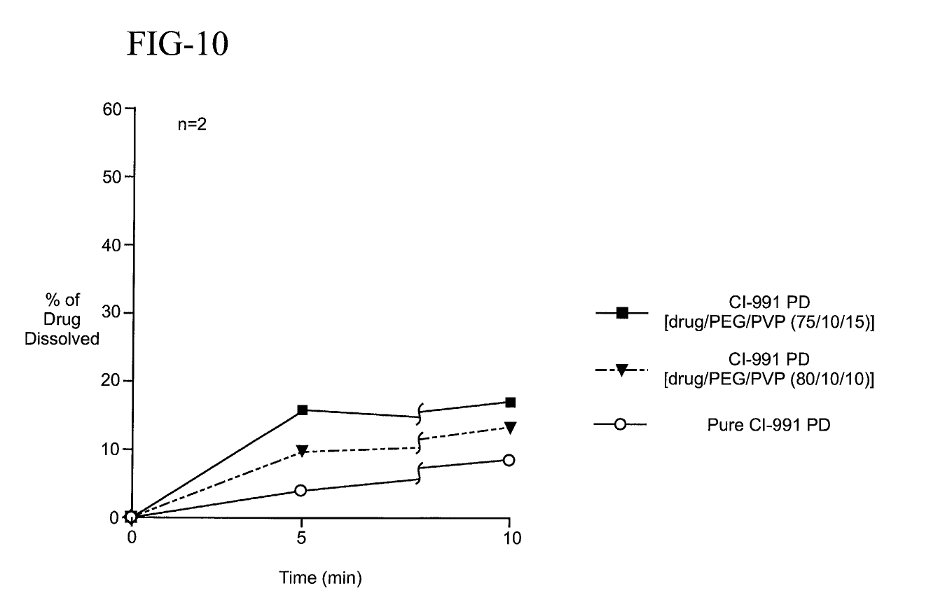

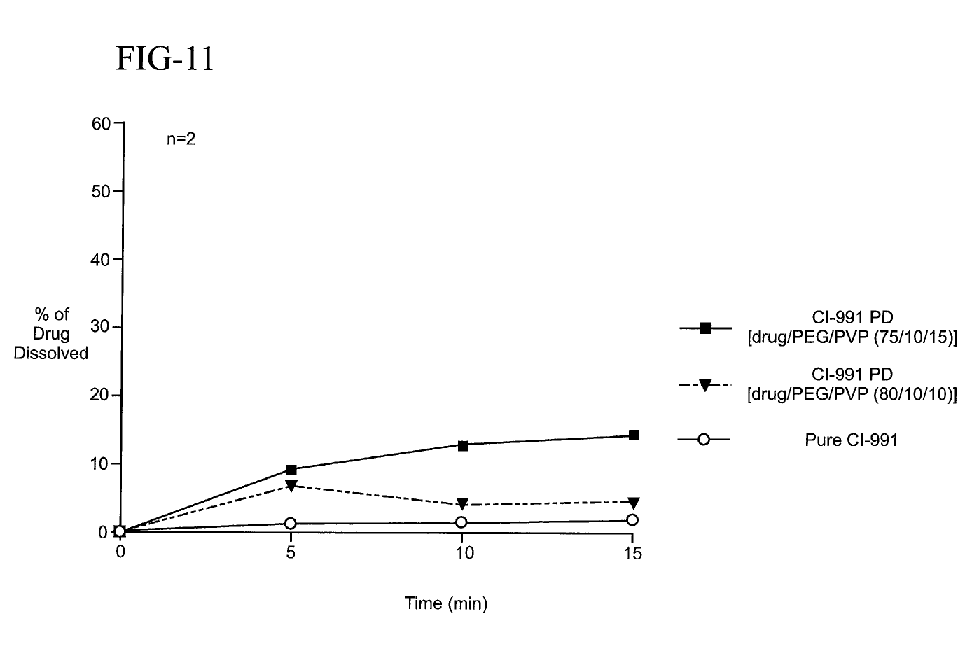

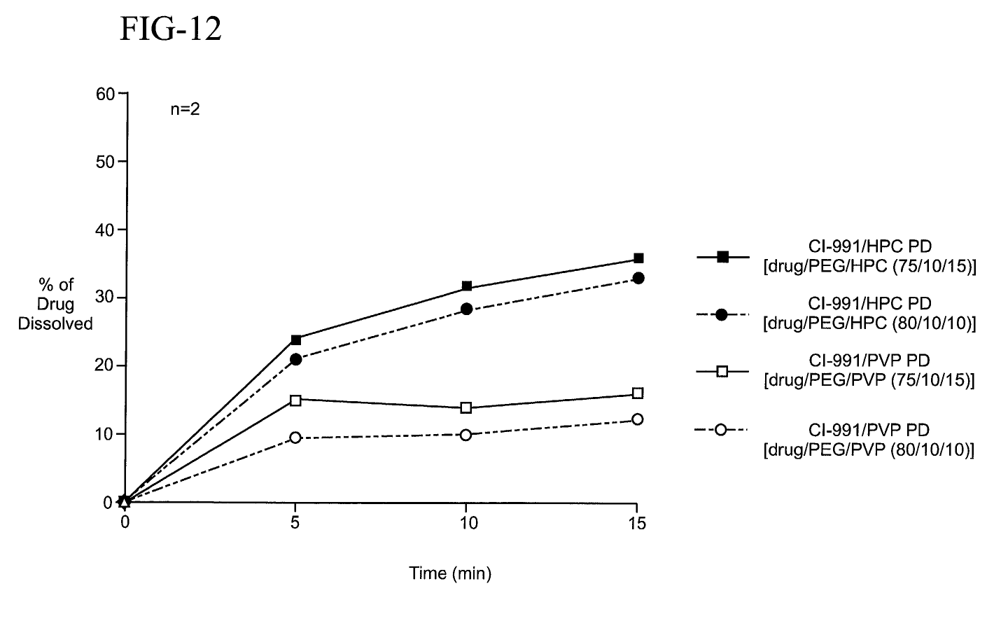

The dissolution profiles of CI-991/PEG-8000/PVP (80:10:10) and (75:10:15) PD in pH 8 (0.1 M) phosphate buffer containing 0.5% SLS and in pH 9 (0.05 M) phosphate buffer are shown in FIGS. 10 and 11, respectively. As with the CI-991/PEG-8000/HPC particulate dispersions, these two CI-991/PEG/PVP PD exhibited faster drug releasing profiles than the pure CI-991. Again, CI-991/PEG/PVP PD with higher concentration of PVP resulted in faster release of drug from the particulate dispersions (FIGS. 10 and 11). These dissolution studies also show that CI-991/PEG/HPC (80:10:10) and (75:10:15) PD have higher dissolution rates than the corresponding CI-991/PEG/PVP PD, especially in pH 8 (0.1 M) phosphate buffer containing 0.5% SLS (FIG. 12). These data obtained may indicate that HPC is a better water-soluble polymer than PVP to enhance the rate of dissolution of drug for CI-991 particulate dispersion. The reason for these differences is not clear yet; however, it may be due to the different physical and chemical properties between HPC and PVP, such as glass transition temperature (Tg), rheological behavior at elevated temperatures, and/or drug-polymer interactions. Nevertheless, this study clearly demonstrated that the rate of dissolution of a poorly water-soluble drugs, CI-991, can be enhanced by the formation of particulate dispersion, in which the drug was coated with (or finely dispersed in) the water-soluble excipients, such as HPC and PVP, at high drug loading.

TABLE 2
Dissolution of Various CI-991/Polymer Particulate Dispersions (PD),
Pure CI-991, and CI-991/HPC (75:25) Physical Mixture in Two
Different Dissolution Media

| Sample | | Percent of CI-991 Dissolved in Solution | | |
|---|---|---|---|---|
| ID | Formulation | at 5 min | at 10 min | at 15 min |

A. In pH 8 (0.1 M) Phosphate Buffer Containing 0.5% SLS

| | | | | |
|---|---|---|---|---|
| TD-0921096 | CI-991/PEG- | 9.5 ± 0.3% | 10.3 ± 0.5% | 12.7 ± 0.6% |
| | 8000/PVP | | | |
| | (80:10:10)PD | | | |
| TD-0931096 | CI-991/PEG- | 21.8 ± 0.5% | 29.2 ± 0.1% | 34.1 ± 0.1% |
| | 8000/PVP | | | |
| | (80:10:10)PD | | | |
| TD-0941096 | CI-991/PEG- | 15.5 ± 2.9% | 14.2 ± 0.4% | 16.7 ± 0.5% |
| | 8000/PVP | | | |
| | (75:10:15)PD | | | |
| TD-0951096 | CI-991/PEG- | 24.9 ± 0.1% | 32.2 ± 0.2% | 36.9 ± 0.2% |
| | 8000/HPC | | | |
| | (75:10:15)PD | | | |
| TD-0961096 | CI-991/PEG- | 38.2 ± 1.9% | 46.2 ± 0.5% | 50.7 ± 0.5% |
| | 8000/HPC | | | |
| | (75:5:20)PD | | | |
| TD-0971096 | CI-991/PEG- | 46.8 ± 3.3% | 51.7 ± 1.6% | 54.9 ± 1.4% |
| | 8000/HPC | | | |
| | (75:25)PD | | | |
| Lot | CI-991 | 3.9 ± 0.1% | 6.3 ± 0.1% | 8.2 ± 0.1% |
| XX020195 | Pure Drug | | | |
| TD-0971096 | CI-991/HPC | 8.3 ± 1.8% | 6.0 ± 0.1% | 7.7 ± 0.1% |
| | (75:25) | | | |
| | Physical | | | |
| | Mixture | | | |

B. In pH 9 (0.05 M) Phosphate Buffer

| | | | | |
|---|---|---|---|---|
| TD-0921096 | CI-991/PEG- | 6.4 ± 0.3% | 4.0 ± 0.4% | 4.7 ± 0.4% |
| | 8000/PVP | | | |
| | (80:10:10)PD | | | |
| TD-0931096 | CI-991/PEG- | 4.9 ± 0.4% | 7.2 ± 0.1% | 8.4 ± 0.1% |
| | 8000/PVP | | | |
| | (80:10:10)PD | | | |
| TD-0941096 | CI-991/PEG- | 8.6 ± 0.1% | 12.6 ± 0.3% | 14.6 ± 0.2% |
| | 8000/PVP | | | |
| | (75:10:15)PD | | | |
| TD-0951096 | CI-991/PEG- | 11.9 ± 1.6% | 11.9 ± 0.1% | 12.5 ± 0.4% |
| | 8000/HPC | | | |
| | (75:10:15)PD | | | |
| TD-0961096 | CI-991/PEG- | 14.9 ± 0.9% | 15.4 ± 0.6% | 16.5 ± 0.2% |
| | 8000/HPC | | | |
| | (75:5:20)PD | | | |
| TD-0971096 | CI-991/PEG- | 24.5 ± 0.4% | 24.6 ± 0.3% | 24.7 ± 0.3% |
| | 8000/HPC | | | |
| | (75:25)PD | | | |
| Lot | CI-991 | 0.5 ± 0.1% | 0.4 ± 0.1% | 1.2 ± 0.2% |
| XX020195 | Pure Drug | | | |
| TD-0971096 | CI-991/HPC | 0.8 ± 0.1% | 1.1 ± 0.1% | 1.3 ± 0.1% |
| | (75:25) | | | |
| | Physical | | | |
| | Mixture | | | |

### Conclusion

Six CI-991/polymer particulate dispersions (PD), namely CI-991/PEG-8000/PVP (80:10:10), CI-991/PEG-8000/HPC (80:10:10), CI-991/PEG-8000/PVP (75:10:15), CI-991/PEG-8000/HPC (75:10:15), CI-991/PEG-8000/HPC (75:5:20) and CI-991/HPC (75:25) PD, were prepared by the mixing bowl method at 110° C. or 130° C. HPLC assay revealed that the drug contents of these particulate dispersions are almost identical to those of theoretical values, suggesting that CI-991 did not undergo significant decomposition during the mixing process at 110° C. or 130° C. X-ray powder diffraction studies suggested that the drug substance in CI-991 particulate dispersions are mostly existed in the crystalline state. The six CI-991 particulate dispersions all exhibited faster drug releasing profiles than the pure CI-991 and CI-991/HPC (75:25) physical mixture in pH 8 (0.1 M) phosphate buffer containing 0.5% (g/mL) SLS and in pH 9 (0.05 M) phosphate buffer. The enhancement of dissolution rate of drug could be mainly due to the increase of wettability and/or the reduction of particle size of CI-991 as the drug was coated with the highly water-soluble polymers such as HPC and PVP during the extrusion process. It is found that HPC appears to be a better water-soluble polymer than PVP to enhance the rate of dissolution of CI-991 from particulate dispersion. This study demonstrated that the rate of dissolution of high dose poorly water-soluble drugs such as CI-991 could be enhanced by improving the wettability of the drugs due to the formation of particulate dispersions.

What is claimed is:

1. A solid particulate pharmaceutical dosage form suitable for oral delivery comprising a sparingly water-soluble particulate pharmaceutical agent dispersed throughout a matrix comprised of a water-soluble polymer.

2. A dosage form of claim 1 wherein the pharmaceutical agent is a glitazone.

3. A dosage form of claim 2 wherein the glitazone is troglitazone.

4. A dosage form of claim 2 wherein the glitazone is BRL 49653.

5. A dosage form of claim 1 wherein the polymer is hydroxypropyl cellulose.

6. A dosage form of claim 1 wherein the polymer is hydroxypropyl methylcellulose.

7. A dosage form of claim 1 wherein the polymer is polyvinylpyrrolidone.
